(19) United States
(12) Patent Application Publication
(10) Pub. No.: US 2015/0258292 A1
(43) Pub. Date: Sep. 17, 2015

(54) DEVICES AND METHODS FOR PREVENTION OF VENTILATOR ASSOCIATED PNEUMONIA

(71) Applicant: The Board of Trustees of the Leland Stanford Junior University, Palo Alto, CA (US)

(72) Inventors: Natalie Stottler, Palo Alto, CA (US); Eric Trac, Hercules, CA (US); Stacie Vilendrer, Palo Alto, CA (US); Prusothman M Sina Raja, Singapore City (SG); Chih-Hao Liu, Taipei City (CN)

(21) Appl. No.: 14/643,815

(22) Filed: Mar. 10, 2015

Related U.S. Application Data
(60) Provisional application No. 61/950,847, filed on Mar. 11, 2014.
(60) Provisional application No. 62/004,227, filed on May 29, 2014.

(51) Int. Cl. A61M 16/04

(52) U.S. Cl. CPC A61M 16/047; A61M 16/0434; A61M 16/0493; A61M 2205/0205

(57) ABSTRACT

Described herein are devices and methods for preventing ventilator associated pneumonia following intubating a patient with an endotracheal tube (ETT). Secretions that accumulate in the trachea above the ETT cuff are wicked out of the patient.

### CROSS-REFERENCE TO RELATED APPLICATIONS

This claims priority to and the benefit of provisional patent application No. 61/950,847, filed Mar. 11, 2014, entitled “Devices and methods for preventing ventilator-associated pneumonia, and to provisional patent application No. 62/004,227, filed May 29, 2014, entitled “Prevention of ventilator-associated pneumonia (VAP),” both of which are incorporated by reference in their entirety.

### BACKGROUND OF THE INVENTION

Mechanical ventilation (MV) is used to partially or fully replace breathing. A ventilator uses positive pressure to force air into the lungs during inhalation, while expiration is passive. MV is required for a variety of patients, including those with lung injuries, respiratory arrest, neurological diseases, or who are just going under anesthesia. For the majority of patients requiring mechanical ventilation, intubation is the primary method. Intubation most commonly involves an endotracheal tube (ETT) inserted through the mouth, although it can also be done through a tracheostomy at the base of the neck or nasal intubation. The “rapid sequence induction” involves pre-oxygenating the patient for 3 minutes, giving a short-acting intravenous sedative and muscle relaxant, using a laryngoscope (a blade with a flashlight used to visualize the tracheal entrance), inserting the endotracheal tube and inflating the “cuff” or peripheral balloon with liquid to keep the tube in place. The tube placement is then checked by either listening for bilateral breath sounds or by x-ray. The attempt should only take about 30 seconds and mechanical ventilation begins immediately after placement. Patients requiring more than 48 hours of intubation are the ones at risk for developing Ventilator Associated Pneumonia, a very serious disease arising from viral or bacterial infection.

There are a few ways in which the presence of the endotracheal tube contributes to VAP. First, the tube prevents the patient from coughing, the reaction that normally helps clear fluid from the airway. Secondly, it allows secretions to accumulate above the cuff and seep through, and lastly, it gives bacteria a surface to grow on, forming what is called a biofilm.

VAP often results from the “microaspiration” of fluids filled with microorganisms that have accumulated above the cuff of the ETT. Although the presence of an endotracheal tube (ETT) can be partially protective against aspiration, it still occurs, because small channels form in the folds of the cuff of the ETT, allowing secretions to slowly leak through the cuff to the lungs. Additionally, the procedure for adjusting the position of the ETT involves deflating the cuff entirely, thus allowing everything that has accumulated above it to fall into the lungs.

Another route for developing VAP is the formation of a biofilm on the surface of the ETT. The biofilm can contain antibiotic-resistant bacteria that develop within hours of intubation. The biofilm itself has a unique structure and community properties that provide an “inherent resistance” to antibiotics. The biofilm has the effect of narrowing the lumen of the ETT and can cause both VAP and ventilator-associated tracheobronchitis. Studies have connected biofilm development with VAP by showing identical microorganisms isolated from the ETT biofilm and tracheal secretions in VAP patients.

Various patents describe strategies to address VAP. For example, U.S. Pat. No. 8,042,544 to Ward et al. (hereinafter referred to as the “the '544 patent”) describes several anti-VAP strategies including use of suctioning to eliminate pooled secretions in the subglottic space. Col 10., line 4. The '544 patent also describes occupying the space with materials. The materials include a foam, liquid, gel, sponge, etc. Col 7., lines 34-47. The '544 patent also describes obliterating the space by injecting various water-soluble hydrogels or foaming agents. Col 10., lines 5-9.

Shortcomings of some of the above mentioned strategies include: an increase in nursing time, patient discomfort, damage to the vocal cords arising from oversized accessories to the ETT, insufficient secretion removal, and added complexity for the intervention. Another drawback of the subglottic secretion drainage (SSD) tube is increased cost. Because the tube is used at the point of initial intubation and reintubation is dangerous, it is used on patients who have no chance of developing VAP.

Notwithstanding the above, an improved construct is desired to mitigate and/or eliminate secretion buildup and biofilm formation above the ETT cuff. An improved construct is desired that prevents VAP associated with an ETT; that conveniently connects to the ETT; that minimizes the complexity and risk during patient intubation; and that is not cost prohibitive.

### SUMMARY OF THE INVENTION

The description, objects and advantages of the present invention will become apparent from the detailed description to follow, together with the accompanying drawings.

A wicking accessory for the prevention of ventilator associated pneumonia when secured to an ETT comprises a flexible substrate adapted to fold around the shaft of the ETT thereby providing a smooth exterior surface lengthwise along the ETT. The substrate includes an enlarged or over-wide distal end section.

The substrate further comprises a plurality of layers including a mucus-wicking layer, and a thin liquid-impenetrable-layer. The liquid-impenetrable layer extends from the proximal end section of the substrate, through the intermediate section, and into the distal end section, leaving at least a portion of the distal end section uncovered by the liquid-impenetrable layer. Mucous in contact with the bare section is drawn via capillary forces through the intermediate section and to the proximal end section.

In another embodiment a wicking apparatus for the prevention of ventilator associated pneumonia comprises an elongate sheet and a capillary structure extending there through to draw mucous in contact with the distal end section through the intermediate section and to the proximal end section. The elongate sheet is adapted to wrap about and conform to an exterior curvature of the shaft of the ETT thereby providing an atraumatic exterior surface along the length of the ETT and locating the distal end section of the sheet in close proximity to the distal cuff member of the ETT. A reservoir is in fluid communication with the proximal end section of the sheet to draw and contain mucous from the proximal end section of the sheet.

In embodiments the capillary structure comprises a network of capillary channels.

In embodiments the capillary structure creates a capillary pressure suitable to draw liquids having a viscosity at least as high as that of mucus present in the trachea of a patient intubated with the ETT.

In embodiments the capillary structure creates a capillary pressure to draw liquid upwards at a first angle, wherein the first angle is at least 35 degrees from horizontal. In embodiments the capillary structure creates a capillary pressure to draw liquid upwards at a flowrate of at least 5 mL/hr.

In embodiments the reservoir has a liquid volume capacity of at least 50 ml. In embodiments the reservoir is dry, and the sheet is wet.

In embodiments the capillary structure comprises an absorbent material. In embodiments the absorbent material is a gauze.

In embodiments the sheet is comprised of a plurality of layers including a first polymeric layer and an absorbent layer.

In embodiments the sheet further comprises a biasing layer facilitating the sheet to bend about a longitudinal axis and inhibiting the sheet to bend about a traverse axis. In embodiments the biasing layer comprises a plurality of ribs extending in the longitudinal direction.

In embodiments the sheet further comprises a hydrophobic layer between the first polymeric layer and the absorbent layer.

In embodiments the first polymeric layer comprises an exterior surface to contact the ETT, and the first polymeric layer is made of a material that provides low friction between the ETT and the exterior surface of the first polymeric layer in both wet and dry environments.

In embodiments the sheet contains an antimicrobial. The antimicrobial may be silver nitrate.

In embodiments the distal end section has a first width, and the intermediate section has a second width, and the first width is greater than the second width. In embodiments the substrate has an unfolded planar configuration in the shape of a dog-bone including the reservoir.

In embodiments the wicking apparatus further comprises a tab to secure the sheet to the ETT.

In embodiments the wicking apparatus further comprises a divider (e.g., a frangible barrier) fluidly separating the reservoir from the proximal end section of the sheet.

In embodiments the wicking apparatus further comprises a bite block located along the proximal end section.

In embodiments the proximal end section, intermediate section, and distal end section of the elongate sheet form one continuous lengthwise-integrated assembly.

In embodiments a VAP prevention assembly comprises an elongate wicking device and a sleeve or appliance attached thereto. The sleeve comprises a first channel for slideably engaging the shaft of the ETT, and a second channel for slideably receiving and guiding the wicking device along the shaft of the ETT and to the ETT cuff member.

In embodiments the sleeve comprises a first arm and a second arm joined to the first arm by a hinge such that the first arm and second arm may be manipulated to form the first channel. In embodiments the first channel has a circular cross section when in a closed configuration.

In embodiments the proximal end section comprises an indicator responsive to the wicking device being saturated with mucous.

In embodiments a method for removing mucous from the trachea of a patient intubated with an ETT comprises wicking the mucous from the trachea, along a plurality of flowpaths, through the vocal folds, and out of the patient's mouth.

In embodiments the wicking is carried out by driving the mucous through an elongate wicking device disposed along the ETT via capillary forces.

In embodiments the method further comprises collecting the mucous outside of the patient in a reservoir fluidly connected to a proximal end of the wicking device.

In embodiments the method further comprises delivering an antimicrobial to the trachea via the wicking device.

In embodiments the method further comprises engaging the wicking device to the ETT in a coaxial arrangement.

In embodiments the method further comprises biasing the wicking device to bend along a longitudinal axis.

### BRIEF DESCRIPTION OF THE DRAWINGS

FIG. 1 is a side view of a sleeve secured to an ETT;

FIG. 2 is an enlarged view of the sleeve and a portion of the ETT shown in FIG. 1;

FIG. 3 is a perspective view of the sleeve shown in FIG. 2 in an unfastened configuration;

FIG. 4 is an illustration of the sleeve shown in FIG. 2 being attached to the ETT;

FIG. 5 is a perspective view of a sleeve secured to an ETT, and guiding a wicking apparatus through the vocal folds;

FIG. 6 is an illustration of a wicking apparatus in an application;

FIG. 7 is side view of an ETT comprising a plurality of lumens, and a wicking apparatus extending through one of the lumens;

FIG. 8 is a side view of the wicking apparatus shown in FIG. 7;

FIG. 9 is a perspective view of another wicking apparatus;

FIG. 10 is an illustration of an ETT and a wicking apparatus secured thereto advanced through the mouth of a patient and into the trachea;

FIG. 11 is a perspective view of a wicking apparatus secured to an ETT, and extending through the vocal folds;

FIG. 12 is an illustration of the wicking apparatus of FIG. 9 shown in an application;

FIG. 13 is a top view of a wicking apparatus in an unfolded configuration;

FIG. 14 is a cross sectional view of the wicking apparatus shown in FIG. 13 taken along line 14-14;

FIG. 15 is an enlarged perspective view of the proximal end of the wicking apparatus shown in FIG. 13;

FIG. 16 is a perspective view of a bite block attached to an ETT;

FIG. 17 is a perspective view of the bite block shown in FIG. 16 in an unfolded configuration;

FIG. 18 is a cross sectional view of a bite block in an application; and

FIG. 19 is a perspective view of an absorption cuff and stiffener member to manipulate the cuff to a target position along an ETT.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

Described herein are apparatuses and methods for preventing VAP by removing secretions that accumulate in the trachea above the ETT cuff. In embodiments, devices and methods are used in combination with the ETT to wick the secretions out of the patient.

Guide Sleeve

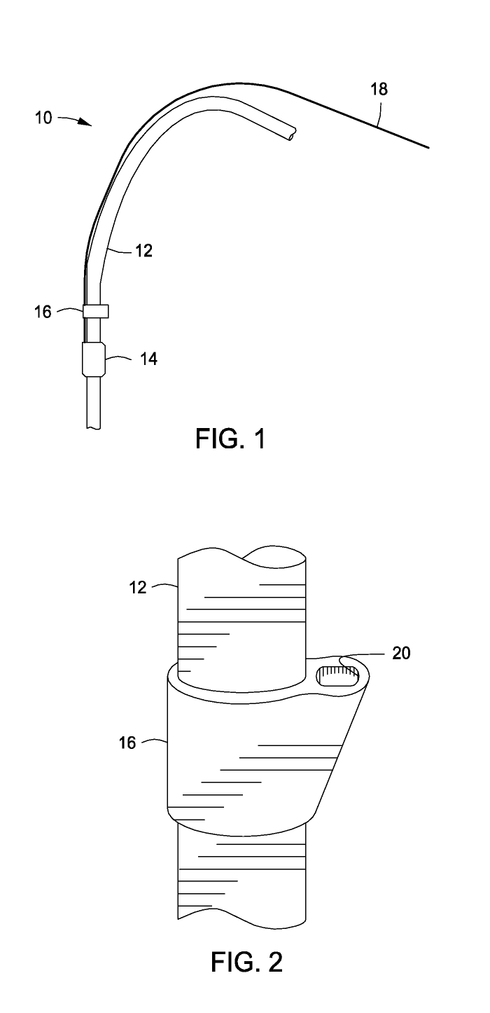

FIG. 1 is a side view of an assembly 10 to prevent VAP by drawing secretions from the trachea via capillary forces. The assembly includes an ETT 12 having a cuff 14, and a sleeve 16 secured to the ETT in a proximal position to the cuff. The sleeve 16 is secured around the ETT 12 such that, following intubation, the sleeve is located just proximal to the vocal folds.

An absorbent wicking apparatus 18 is inserted through the sleeve 16, and towards the cuff 14 of the ETT.

The wicking apparatus 18 shown in FIG. 1 is elongate and narrow. It is small enough to fit through open areas between vocal folds. An exemplary outer diameter for the wick 18 ranges from 1 to 3 mm, and preferably is about 2 mm. The length of the wick may also vary. In embodiments, the length ranges from 15 to 40 cm, and preferably is 35 cm., which can provide about 1 cm³ of absorbent material.

Absorbent Material

The wicking apparatus is preferably hyper-absorbent and able to transport secretions by capillary forces against gravity. Preferable characteristics for the material include, without limitation, a material which can absorb more than 100% of its weight in water, or one having a density of about 1.51 g/cm³. Examples of materials include viscose rayon fibers and gauze made from Rayon, Polyester, Cellulose blends.

The wicking apparatus may also be loaded or dampened with bioactive agents. In embodiments, the wicking apparatus may deliver medicines, therapeutics, antibiotics, antimicrobials, or drugs to the tissue. In one embodiment, the wick is dampened with, and can elute, ionized silver for its antimicrobial properties.

The wicking apparatus may have a plurality of layers or coatings. For example, a low friction coating (such as Teflon) can be disposed on at least a portion of the device. A distal section is left uncovered by the low friction layer. In an embodiment, all but the last 3 cm of absorbent material of the wicking apparatus is covered. The low friction coating facilitates gliding the wicking apparatus through the sleeve as well as through the vocal folds. The diameter of the end section or bare section can be less than the diameter of the intermediate section. The bare section of the wicking apparatus may be about 1.8 mm.

FIG. 2 is an enlarged view of the sleeve and a portion of the ETT shown in FIG. 1. The sleeve has an inlet or guide lumen 20 through which the absorbent wick 18 is inserted. The cuff 16 may be made of a polymer such as PET. A low friction coating (such as Teflon) may be applied to the sleeve body to aid in positioning.

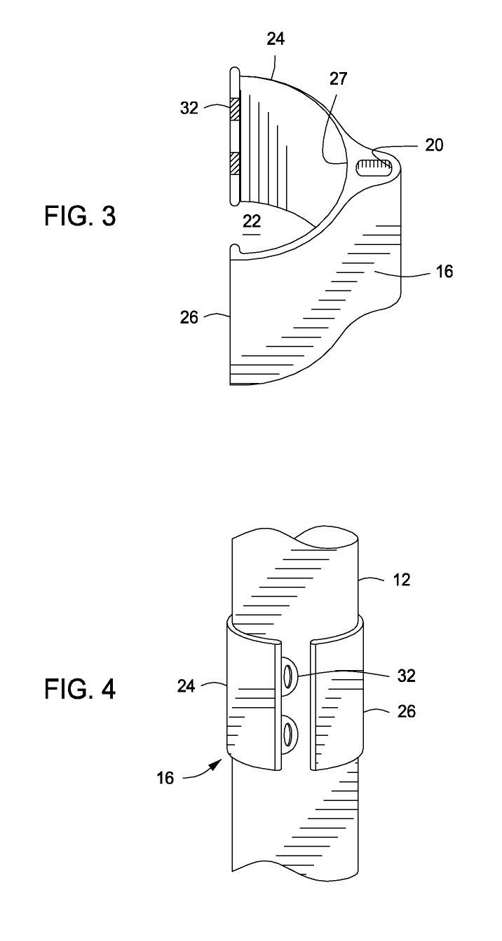

FIG. 3 is a perspective view of the sleeve 16 in an unfastened configuration. Sleeve 16 comprises a living hinge 27 at its center to support a plurality of arm members 24, 26. The arms define a space or cavity 22 which can accept the shaft of the ETT, of the size for which the sleeve is rated.

FIG. 4 is an illustration of the sleeve 16 being attached to the ETT 12. Arm members 24, 26 and hinge cooperate to receive the ETT 12 and to snap around the ETT. Two buckles 32 snap into place as the cuff is closed around the ETT 12. Although buckles are illustrated, other fasteners or interlocking elements may be utilized.

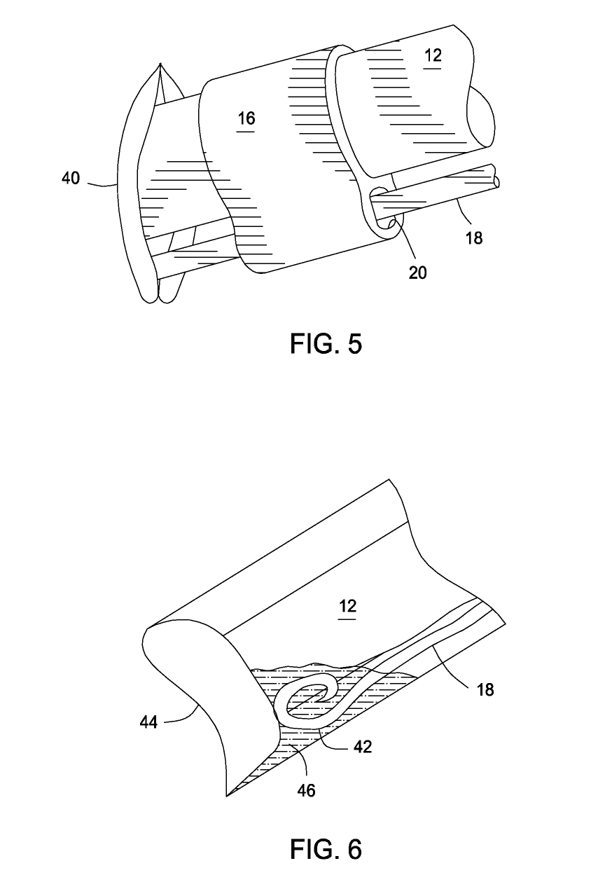

After the sleeve 16 is positioned around the shaft of the ETT 12, a respiratory therapist or nurse inserts the wicking apparatus 18 into the guide hole 20. Initially, the guide hole should be positioned on the top of the ETT, just above the vocal folds. The nurse then attempts to feed the wicking apparatus 18 through the vocal folds 40. If she is met with resistance, the rotation of the guide cuff should be adjusted. This adjustability allows the device to be used on patients with variations in shape and size of the vocal folds. FIG. 5 is an illustration of a wicking apparatus 18, guided by sleeve 16 through the vocal folds 40.

With reference to FIG. 6, the nurse then feeds the wicking apparatus 18 all the way in, to the point where the visible end of the wicking apparatus is at the end of the ETT and can contact liquids 46.

Because the wicking apparatus is flexible and the distal section lacks the support of the outer polymer layer described above, the distal section of the wicking apparatus can fall softly around the ETT cuff 44, thus reaching more secretions 46. Additionally, because mechanical ventilation patients are typically positioned at a 30 degree angle, secretions tend to pool on the back side of the trachea where the wick will be positioned.

In embodiments, the extracorporeal end of the wick includes an indicator allowing the physician, nurse or respiratory therapist to observe when the wicking apparatus is ready to be replaced. When the indicator is wet, for example, a color change may be observed. In one embodiment, the extracorporeal end of the wick attaches to a spiral rayon thread of 30 cm³. The wick is ready to be replaced when the spiral end of rayon thread becomes wet, indicating a threshold or certain volume (e.g., 50 mL) of secretions have been absorbed. The indicator may be formed of other materials including without limitation polyvinyl alcohol.

Methods include replacing the wicking apparatus. For example, the wicking apparatus may be replaced twice a day, as patients can produce daily subglottic secretions ranging from as low as a few milliliters to as high as 100 mL.

The entire process to install the sleeve on the ETT, and insert the wicking apparatus into position is estimated to take less than 5 minutes, with replacement wicks requiring even less time because the guide indicates the ideal insertion area, so as not to significantly increase nursing requirements.

Multi-Lumen Endotracheal Tube

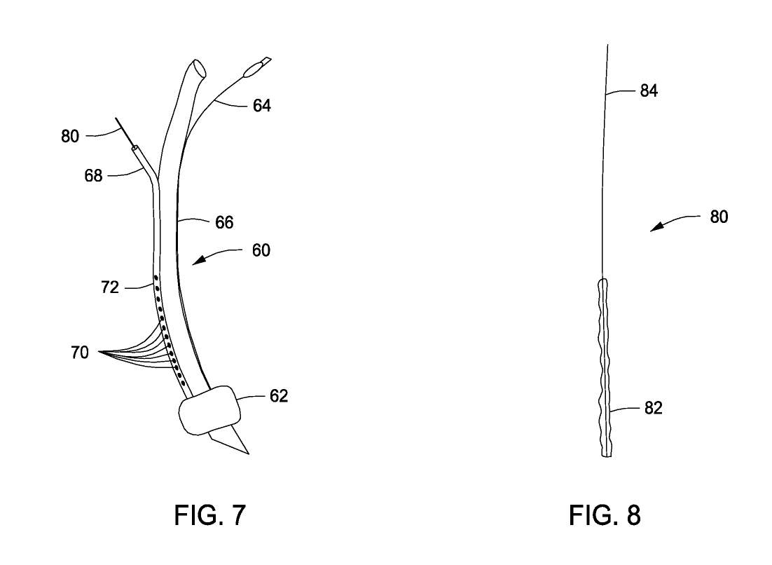

FIGS. 7 and 8 illustrate another strategy for preventing VAP by removing secretions from the trachea.

FIG. 7 is side view of an improved ETT 60 comprising a first lumen 66, a second lumen 68 and a wicking apparatus 80 extending through the second lumen. The wicking apparatus 80 is shown advanced through a second lumen 68 of the ETT 60, and into an elongate chamber 72. The chamber 72 comprises a plurality of holes or pores to accept liquid from the trachea. It should be noted that the chamber construct may vary in shape and location. For example, in another embodiment, the chamber is disposed outside the main tube body rather than inside it. Additionally, the chamber may have a flexible or stiff wall.

The proximal end of the wicking apparatus exits the ETT, which would be just above the mouth area of a patient following intubation. However, the invention is not so limited. In other embodiments, the second lumen 68 exits the ETT within the mouth, further up from the mouth, or through a port on the connector to the mechanical ventilator.

The multi-lumen ETT tube 60 has functionality similar to a standard ETT tube, but in addition, has the chamber 72 that connects to the outside of the cuff area via a series of pores 70. The pores 70 allow secretions to fill the chamber 72.

FIG. 8 illustrates the wicking apparatus 80 shown in FIG. 7. The wicking apparatus generally includes an elongate shaft 84 having a distal section 82 comprising liquid wicking capability.

Distal section 82 may comprise an absorbent material, preferably a hyper-absorbent material for wicking secretions by capillary forces as described herein. The wand 80 passively absorbs secretions through capillary action. Examples of materials for the absorbent material in the wicking apparatus include, without limitation, a sponge material, an absorbent polymer such as sodium polyacrylate, PVA (Poly Vinyl Alcohol), and a chamois. The materials may be fastened to the shaft by adherence, dipped, fused, or otherwise attached to the shaft. In embodiments, and as described further herein, the shaft itself may be made of an absorbent material or composite structure having absorbent properties and sufficient rigidity for manual manipulation by a physician, nurse or respiratory therapist.

Wicking apparatus 80 may be replaced when saturated or full. In embodiments, the wand 80 is replaced when an indicator, as described above, on the wand changes colors. Because the system is passive, unlike suctioning, very little nursing time is required and patient discomfort is minimized.

Wicking Sheet

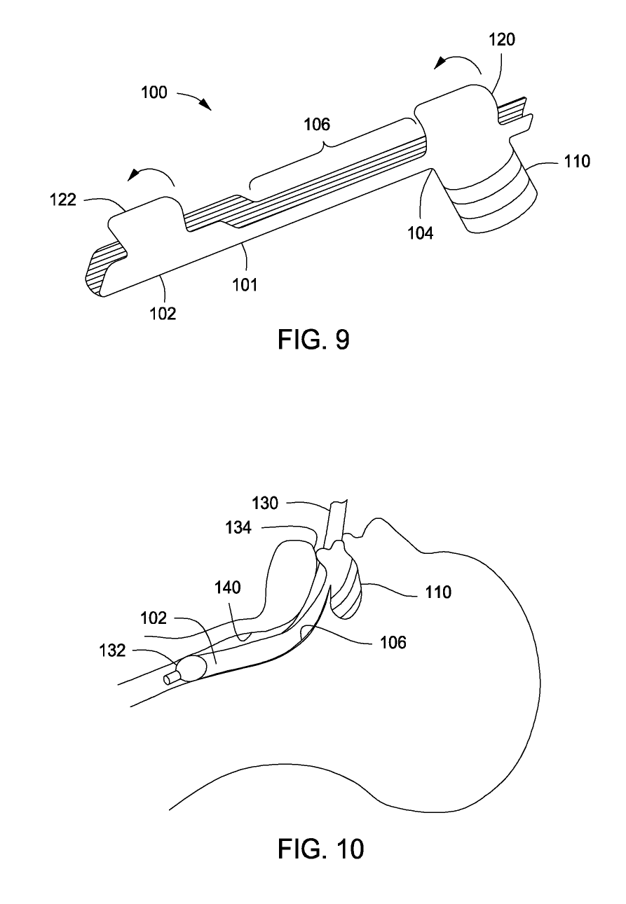

FIG. 9 illustrates a wicking apparatus 100 intended to wick secretions from the space above the ETT (e.g., the subglottic space) to outside the patient. The wicking apparatus 100 may be provided in the form of an elongate absorbent wicking sheet 101, and optionally, a reservoir 110 for secretion storage outside the patient.

FIG. 10 is an illustration of an ETT 130 and a wicking sheet 100 secured thereto. The wicking sheet 101 wraps about (or circumferentially surrounds) the exterior of the ETT 130 such that the distal end section 102 of the wicking apparatus is fixed proximal and adjacent to the cuff 132 of the ETT.

The coaxial arrangement of the ETT and wicking apparatus shown in FIG. 10 allows the assembly to be advanced through the patient's mouth 134 and into the trachea 140 simultaneously.

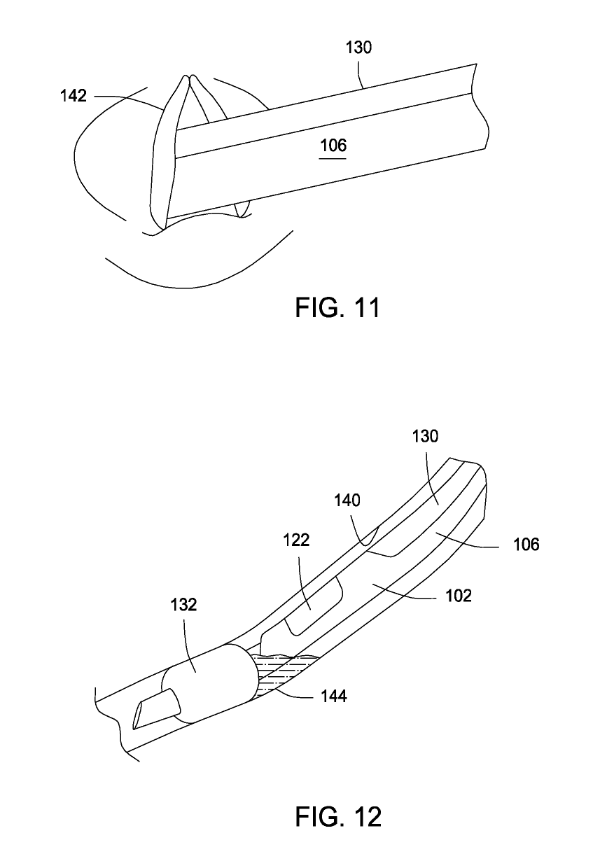

FIG. 11 is a partial perspective view of a portion of the wicking apparatus 106 secured to the ETT 130, and extending through the vocal folds 142.

FIG. 12 is an illustration of the wicking apparatus 100 in an application to draw or wick secretions 144 from the trachea 140. Distal end section 102 of the wicking apparatus has been wrapped and fastened to the ETT shaft 130 by adhesive tab 122. As shown, the wicking sheet is in a location to contact secretions 144 building up at the cuff 132. Liquids to contact the wicking sheet are drawn and wicked upwards through the wicking sheet, and out of the patient's mouth. As discussed further herein, a reservoir 110 may be provided to facilitate liquid removal and collection.

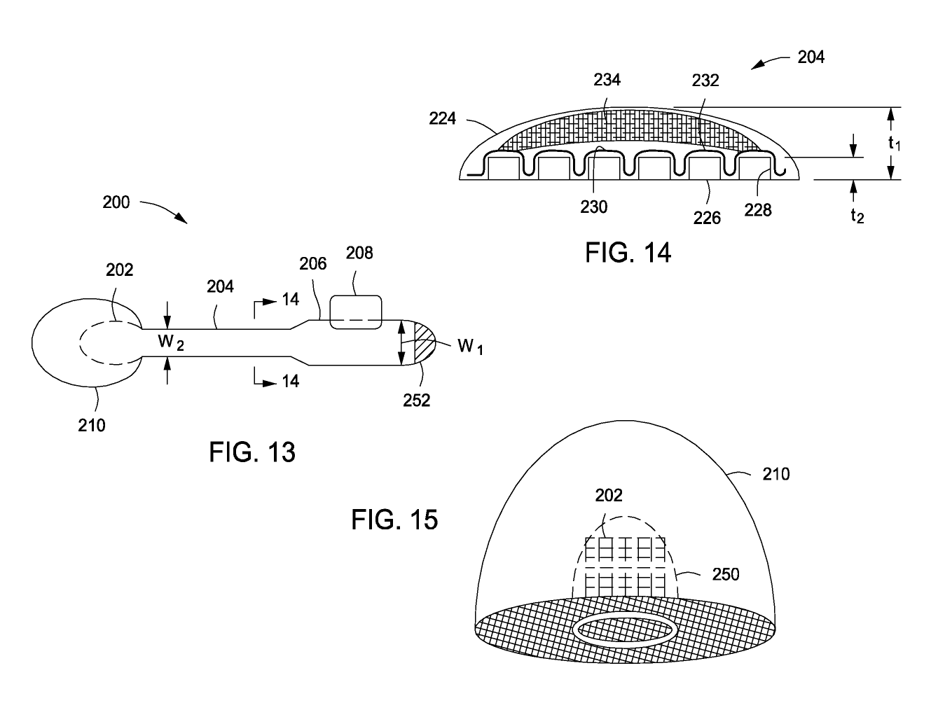

FIG. 13 is a top view of another wicking apparatus 200. The wicking apparatus 200 comprises a proximal end section 202, intermediate section 204, and a distal end section 206. As mentioned above, a tab or seal 208 may be used to fasten or secure the wicking apparatus to the ETT. An adhesive (e.g., a silicone-based adhesive) with a perforation on one side can be used to hold the cuff/ETT together.

An optional reservoir 210 for secretion storage is shown surrounding or in fluid communication with the proximal end section of the wicking apparatus 200.

FIG. 14 is a cross sectional view of the wicking apparatus shown in FIG. 13 taken along line 14-14.

As shown, the wicking sheet or substrate 204 comprises a plurality of layers collectively forming an overall thickness (t₁) ranging from 0.5 to 2 mm, preferably 1 mm. However, the ranges and features provided herein are intended as exemplary and the invention is not intended to be so limited except as where specifically recited in the appended claims.

First layer 226 is a flexible layer on the side that touches the ETT shaft. An example of the first layer is a polyethylene film. A low-friction material (such as polyethylene) is desirable because it provides low friction on the ETT in both wet and dry environments.

A set of rib elements 228 are shown on top of the first layer 226. The ribs extend longitudinally along the wicking apparatus. Preferably, ribs are about 0.5 mm in height (t₂). An exemplary material for the ribs is polycarbonate. The ribs serve to makes the device stiff in one direction, but flexible in another direction (e.g., to bend/wrap around the ETT tube).

A hydrophobic treatment or coating 232 may be applied to the ribs and top layer. Examples of a hydrophobic coating include a silicone-based or parylene application.

An absorbent layer 234 is shown on top of the ribs 228. As described herein, a wide variety of materials or compositions may be used for the absorbent layer and to provide wicking forces. An example of an absorbent layer is a gauze made from a Rayon, Polyester, Cellulose blends such as the Quick-Loc™ Gauze (manufactured by Johnson & Johnson, Inc., New Brunswick, N.J.). In embodiments, the gauze is dampened with antimicrobial agents which are released to the trachea. An exemplary antimicrobial agent is 0.1% silver nitrate.

A top layer 224 is shown enclosing the gauze. The top layer 224 may be formed of a thin flexible low-friction material such as a polyethylene film. The gauze-polyethylene combination described above serves to make the device flexible, soft, and less prone to damaging the vocal folds.

It should be noted, however, the outer layers are not required to extend the entire length of the wicking apparatus 200. In embodiments, a bare portion 252 of the distal end section 206 is not covered with one or more of the outer layers so as to allow secretions to enter the wicking device.

Reservoir

FIG. 15 is an enlarged perspective view of the proximal end section 202 of the wicking apparatus 200. A reservoir 210 in the shape of a pouch surrounds the proximal end 202. The volume capacity of the reservoir preferably ranges 35 mL to 120 mL. Or, in embodiments, the reservoir is at least large enough to be used for 24 hours before replacement.

The reservoir 210 preferably comprises absorbent material to absorb wicked secretions from the proximal end section of the wicking sheet. The reservoir absorber may be the same as that used in the wicking sheet. For example, the reservoir material may comprise a gauze. Another suitable material for the reservoir to absorb the wicked secretions is sodium polyacrylate. The sodium polyacrylate may also be used in combination with a gauze.

Also, in embodiments in which the wicking substrate is provided damp (e.g., loaded with an antimicrobial agent) and the reservoir is provided dry, a divider 250 can separate the two components prior to use. The physician, nurse or respiratory therapist punctures or breaks the divider to activate, allowing the damp wicking sheet 202 to meet the absorbent material within the reservoir 210. An example of a divider and pouch assembly is a disposable cold pack, Dynarex Instand Cold Packs (manufactured by Dynarex Corporation, Orangeburg, N.Y.).

The shape or construct of the reservoir may vary. In another embodiment, the reservoir is a container (e.g., bottle, vial, tube, etc.). It may be rigid or soft. The container may comprise absorbent materials as described above in connection with reservoir 210.

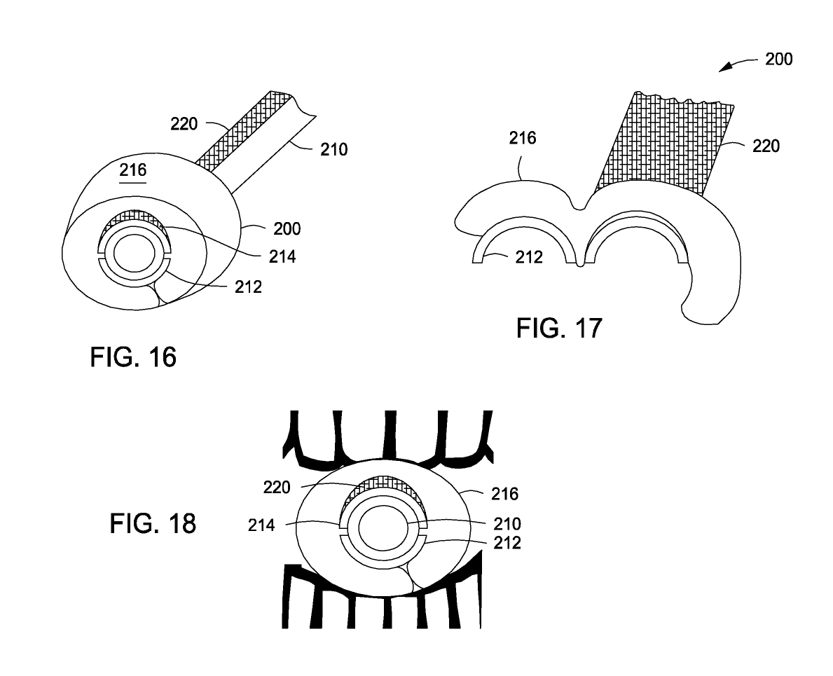

FIG. 16 is a perspective view of a bite block 200 attached to the proximal end section of an ETT assembly 210. The bite block 200 is shown having an inner hard plastic layer comprised of a plurality of sections (namely, two semicircles 212, 214). Bite block 200 also includes a soft exterior 216. Materials for the soft exterior layer 216 include without limitation silicone and rubber.

In embodiments, the bite block 200 includes an adhesive 220 for bonding the bite block to the ETT assembly 210.

With reference to FIG. 17, bite block may be provided in an unfolded configuration and installed over the proximal end section of the ETT assembly by closing the bite block over the ETT assembly.

FIG. 18 is a cross sectional view of a bite block in an application, namely, being compressed by teeth 230 in the mouth of a patient. Teeth 230 are shown compressing the outer layer but do not damage or significantly affect the operation of the ETT and associated wicking apparatus discussed herein. The bite block serves to protect the wicking apparatus and ETT.

### EXAMPLES

Wicking devices as described above in connection with FIG. 9 were tested for wicking power in both saliva and mucous wicking. The length of the wicking sheet was 17 cm. The devices and saliva/mucus were placed in an insulated box maintained at temperature between 96 and 99 degrees F. Each device was placed at an angle of about 40 degrees from horizontal. Thus, the wicking devices were required to draw liquids against the force of gravity.

Two trials were performed on mucus. The duration of the mucus test was 7 hours. The mucus used was 3-day old nasal mucus harvested from a patient with a cold. The average volume of mucus wicked was 36 mL, stdev: 0.35. mL. This corresponds to a flowrate Q_{M} of about 5 mL/hour.

Three trials were performed on saliva. The saliva test was also 7 hour wicking challenge test. The saliva was fresh, having been generated by a healthy subject in the 24 hours preceding the trial. The average volume of saliva wicked was 40 mL. This corresponds to a flowrate Q_{S} of about 6 mL/hour.

The above examples demonstrate wicking strength of devices described herein. Such devices are capable and structured to draw liquids having viscosities equal to that of saliva and mucus. And, the viscosity of saliva is known to be up to about 23 cP at low shear rates (e.g., 2.25-450 1/sec), and as low as about 3 cP at higher shear rates (e.g., 0.5-94.5 1/sec). See Rantonen, Panu J F, and Jukka H. Meurman. “Viscosity of whole saliva.” Acta Odontologica 56.4 (1998): 210-214.). See also Preetha, A., and R. Banerjee. “Comparison of artificial saliva substitutes.” Trends Biomater Artif Organs 18.2 (2005): 178-186.)

Despite the relatively high viscosity of the liquids tested, and despite opposing gravitational forces due to the upwards testing angle, the wicking test devices transported the liquids from the distal end section to the proximal end section and into the reservoir. This is clear support for the surprising wicking power and capillary strength of the invention described herein.

### Other Embodiments

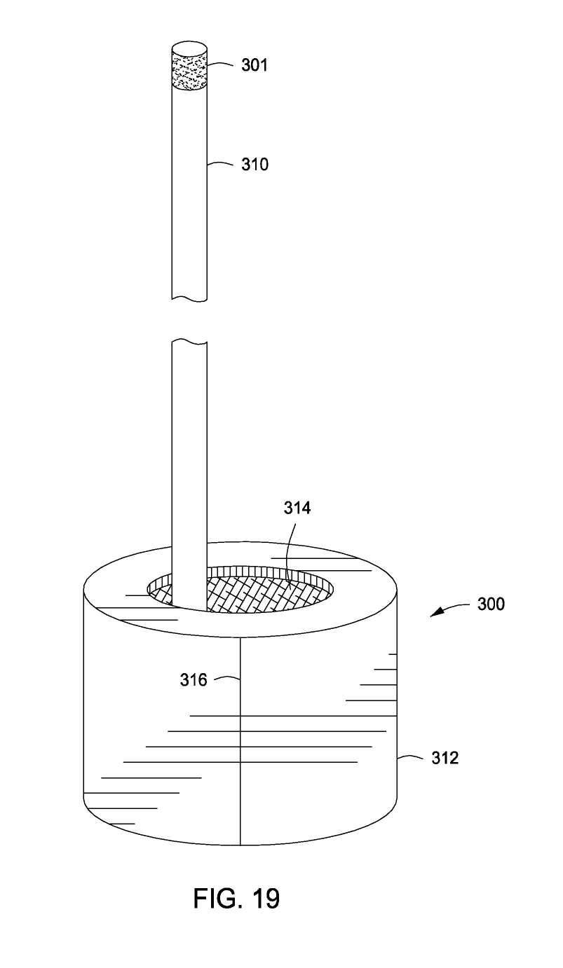

FIG. 19 is a perspective view of an absorption cuff 300 to be secured to the exterior of an ETT. After the cuff 300 is secured, the user manipulates a thick wire 310 in order to slowly guide the cuff along the tube to the subglottic area just above the cuff of the ETT. Capillary action works to passively absorb secretions as the absorption cuff is left in place. In embodiments, an indicator 301 at the proximal end of the wire guide changes color to show that the absorption cuff has reached its liquid limit at which time, the absorption cuff is replaced by another one. Because this absorption is passive, the cuff reduces nurse time as compared to suctioning.

As shown, the absorption cuff 300 has a cylinder shape 312. It may be made of hyper-absorbent material supported by a thin wire frame 314. A thicker wire 310 extends from the wire frame/cylinder. The stiffener 310 aids in the placement of the cuff. It is intended for use in the ICU 24 hours after initial intubation. The cuff 300 is shown having a septum 316 for opening and to allow its initial placement around the tube.

The cuff 300 may be secured using a variety of methods including, for example, Velcro and magnets. The cuff may be made from a wide variety of materials including but not limited to a sponge material, an absorbent polymer (such as, e.g., sodium polyacrylate), Poly Vinyl Alcohol, and a porous cloth (such as a porous leather or chamois).

In another embodiment, an elongate device (e.g., a catheter) sterilizes the internal biofilm of an ETT by directing UV light at the biofilm. In another embodiment a heating apparatus or accessory is attached to the ETT. The heating apparatus warms and cleanses the ventilated air of most VAP-causing bacteria. Embodiments of such devices and catheters are shown and described in provisional patent application No. 61/950,847, filed Mar. 11, 2014, entitled “Devices and methods for preventing ventilator-associated pneumonia, and to provisional patent application No. 62/004,227, filed May 29, 2014, entitled “Prevention of ventilator-associated pneumonia (VAP), both of which are incorporated by reference in their entirety.

While preferred embodiments of this disclosure have been shown and described, modifications thereof can be made by one skilled in the art without departing from the scope or teaching herein. The embodiments described herein are exemplary only and are not intended to be limiting. Because many varying and different embodiments may be made within the scope of the present inventive concept, including equivalent structures, materials, or methods hereafter thought of, and because many modifications may be made in the embodiments herein detailed in accordance with the descriptive requirements of the law, it is to be understood that the details herein are to be interpreted as illustrative and not in a limiting sense.

We claim:

1. A wicking apparatus for the prevention of ventilator associated pneumonia when used with an ETT, the ETT comprising an elongate shaft and a distal cuff member, the wicking apparatus comprising:
an elongate sheet comprising a proximal end section, an intermediate section, and a distal end section, and a capillary structure extending there through to draw mucous in contact with the distal end section through the intermediate section and to the proximal end section, said elongate sheet further adapted to wrap about and conform to an exterior curvature of the shaft of the ETT thereby providing an atraumatic exterior surface along the length of the ETT and locating the distal end section of the sheet in close proximity to the distal cuff member of the ETT; and
a reservoir in fluid communication with the proximal end section of the sheet to draw and contain mucous from the proximal end section of the sheet.

2. The wicking apparatus of claim 1 wherein the capillary structure comprises a network of capillary channels.

3. The wicking apparatus of claim 2 wherein the capillary structure comprises an absorbent material.

4. The wicking apparatus of claim 3 wherein the absorbent material is a gauze.

5. The wicking apparatus of claim 1 wherein the sheet is comprised of a plurality of layers, said plurality of layers comprising a first polymeric layer, and an absorbent layer.

6. The wicking apparatus of claim 5 wherein the sheet further comprises a biasing layer facilitating the sheet to bend about a longitudinal axis and inhibiting the sheet to bend about a traverse axis.

7. The wicking apparatus of claim 6 wherein the biasing layer comprises a plurality of ribs extending in the longitudinal direction.

8. The wicking apparatus of claim 6 further comprising a hydrophobic layer between the first polymeric layer and the absorbent layer.

9. The wicking apparatus of claim 1 wherein the capillary structure creates a capillary pressure suitable to draw liquids having a viscosity at least as high as that of mucus present in the trachea of a patient intubated with the ETT.

10. The wicking apparatus of claim 1 wherein the capillary structure creates a capillary pressure to draw liquid upwards at a first angle, wherein the first angle is at least 35 degrees from horizontal.

11. The wicking apparatus of claim 10 wherein the capillary structure creates a capillary pressure to draw liquid upwards at a flowrate of at least 5 mL/hr.

12. The wicking apparatus of claim 1 wherein the reservoir is dry, and the sheet is wet.

13. The wicking apparatus of claim 12 further comprising a frangible barrier fluidly separating the reservoir from the proximal end section of the sheet.

14. The wicking apparatus of claim 1 wherein the sheet comprises an antimicrobial.

15. The wicking apparatus of claim 14 wherein the antimicrobial comprises silver nitrate.

16. The wicking apparatus of claim 1 wherein the distal end section has a first width, and the intermediate section has a second width, and the first width is greater than the second width.

17. The wicking apparatus of claim 1 further comprising a bite block located at the proximal end section.

18. The wicking apparatus of claim 5 wherein the first polymeric layer comprises an exterior surface to contact the ETT, and the first polymeric layer is made of a material that provides low friction between the ETT and the exterior surface of the first polymeric layer in both wet and dry environments.

19. The wicking apparatus of claim 5 wherein the proximal end section, intermediate section, and distal end section of the elongate sheet are lengthwise integrated.

20. A wicking accessory for the prevention of ventilator associated pneumonia when secured to an ETT, the ETT comprising an elongate shaft and a distal cuff member, the wicking accessory comprising:
a flexible substrate comprising a proximal end section, an intermediate section, and a distal end section,
wherein said elongate flexible substrate is flexible to fold around an exterior of the shaft of the ETT thereby providing a smooth exterior surface lengthwise along the ETT and locates the distal end section of the accessory in close proximity to the distal cuff member;
wherein the distal end section is wider than the intermediate section; and
wherein the substrate further comprises a plurality of layers including a mucus-wicking layer, and a thin liquid-impenetrable-layer, said liquid-impenetrable layer extending from the proximal end section of the substrate, through the intermediate section, and into the distal end section and leaving at least a portion of the distal end section uncovered by the liquid-impenetrable layer such that mucous in contact with the uncovered distal end section is drawn via capillary forces through the intermediate section and to the proximal end section.

21. A method for preventing VAP comprising passively removing subglottic secretions from the trachea.

22. The method of claim 21 wherein the passively removing step comprises wicking the mucous from the trachea, along a plurality of flowpaths, through the vocal folds, and out of the patient's mouth.

23. The method of claim 21 wherein the removing step is performed subsequent to initial intubation of the patient with an ETT.
